(19) United States
(12) Patent Application Publication
(10) Pub. No.: US 2024/0090598 A1
(43) Pub. Date: Mar. 21, 2024

(54) SURGICAL GOWNS

(71) Applicant: Mayo Foundation for Medical Education and Research, Rochester, MN (US)

(72) Inventors: Salim M. Walji, Albuquerque, NM (US); Joseph A. Dearani, Rochester, MN (US)

(21) Appl. No.: 18/524,497

(22) Filed: Nov. 30, 2023

Related U.S. Application Data
(63) Continuation of application No. 17/056,581, filed on Nov. 18, 2020, now Pat. No. 11,857,002.
(60) Provisional application No. 62/677,843, filed on May 30, 2018.

(51) Int. Cl. A41D 13/00; A41D 13/12

(52) U.S. Cl. CPC A41D 13/0012; A41D 13/1209

(57) ABSTRACT

Surgical gowns can be configured to be worn by a human and to be sterile for use in a sterile surgical environment. Such a sterile surgical gown can include one or more pockets attached to the gown and configured to store surgical instruments. The sterile surgical gown can also include one or more rings attached to the gown and configured to hold surgical instruments. In some embodiments, a patch that is attachable to the gown. The patch can include one or more additional pockets or additional rings.

### CROSS-REFERENCE TO RELATED APPLICATIONS

This application is a continuation of U.S. application Ser. No. 17/056,581 filed on Nov. 18, 2020, which is a National Stage application under 35 U.S.C. § 371 of International Application No. PCT/US2019/034570 having an International Filing Date of May 30, 2019, which claims priority to U.S. Application Ser. No. 62/677,843 filed on May 30, 2018. The disclosure of the prior applications are considered part of the disclosure of this application, and are incorporated in their entireties into this application.

### BACKGROUND

### 1. Technical Field

This document relates to improved designs of surgical gowns.

### 2. Background Information

During surgery, the Certified Scrub Technician (CST) is generally focused on the needs of the Primary Surgeon (“Surgeon”), and is busy passing instruments to the Surgeon. The needs of the “Assistants” (Assistant Surgeon/Resident/Certified Surgical Assistant) on the other side of the surgical table are legitimate but sometimes can be a distraction from the CST's point of view. From the Assistant's point of view, any delay in getting the necessary instruments can sometimes be frustrating and hinder the smooth running of the procedure.

### SUMMARY

This document describes improved designs of surgical gowns.

In one implementation, this disclosure is directed to a surgical gown configured to be worn by a human and to be sterile for use in a sterile surgical environment. Such a sterile surgical gown can include one or more pockets attached to the gown and configured to store surgical instruments. The sterile surgical gown can also include one or more rings attached to the gown and configured to hold surgical instruments. In some embodiments, a patch that is attachable to the gown. The patch can include one or more additional pockets or additional rings.

Particular embodiments of the subject matter described in this document can be implemented to realize one or more of the following advantages. In some embodiments, the surgical gowns described herein include pockets that can conveniently contain basic instruments such as scissors and forceps. These pockets can be located at different locations and varying angles to facilitate optimal access and thereby greatly obviate the need for almost 30-80% of the back and forth moves that occur between an Assistant and the CST.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of a clinician wearing an example surgical gown in accordance with some embodiments.

Like reference numbers represent corresponding parts throughout.

### DETAILED DESCRIPTION

This document describes improved designs of surgical gowns.

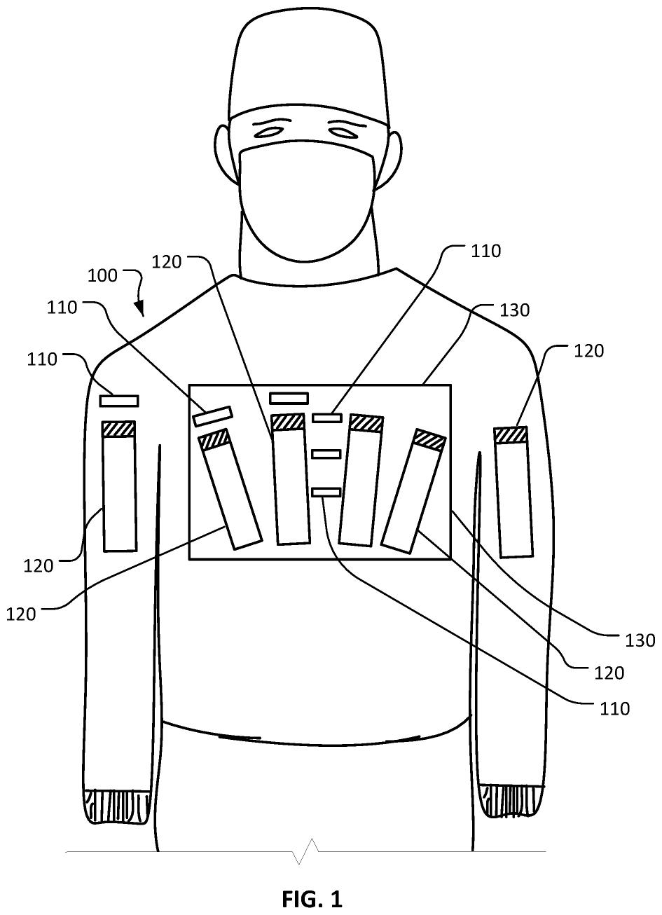

A surgical gown 100 with some “pockets” as anchors (such as exemplified in FIG. 1), could provide literally a storage area that could provide the Assistants rapid access to their needs (forceps/scissors or such small items) and greatly obviate the need for almost 30-80% of the back and forth moves that occur between an Assistant and the CST. The inventors believe this would greatly improve the efficiency during a surgical procedure.

The inventive concepts and designs of the surgical gowns described herein may also have broader applicability in other areas of clinical and laboratory medicine as well as other industries beyond medicine—where convenience and improved efficiency are worthy and desired goals.

Referring to FIG. 1, the example surgical gown 100 can include one or more holsters, rings, and/or belt loops (collectively referred henceforth as “loops 110”) and one or more pockets 120. In some embodiments, the loops 110 and/or the pockets 120 are both made of flexible material such as fabric.

The loops 110 and/or the pockets 120 are designed to hold instruments such as forceps/pickups and scissors or other simple, narrow instruments (the “instruments”). In some embodiments, these surgical gowns 100 are disposable—unlike the cloth gowns. In some embodiments, the gowns 100 can be reusable.

The size (width and length), position and angle of the pockets 120 and/or loops 110 can be selected as needed for optimal access, ease and sterility.

The loops 110 are somewhat similar in configuration as those on trousers that hold belts (belt loops). These can be in place of the pockets 120, or supplement the pockets 120 as additional guides and anchors for the instruments. The loops 110 can be made in various sizes. A single gown 100 can have loops 110 of various sizes. In some embodiments, the loops 110 are made of an elastic. In some embodiments, two or more loops 110 are arranged in alignment with each other so that a single instrument will be held by the two or more aligned loops 110.

In some embodiments, the pockets 120 can be free-standing by themselves or be supplemented with the loops 110, as mentioned above—or in various combinations. The goals of the pockets 120 and loops 110 are to hold the instruments and to promote easy and rapid access to the instruments for the surgeon and surgical assistants. This can greatly promote the surgical efficiency at multiple levels during a procedure for the entire surgical team.

The instruments are secured in a stable fashion, e.g., to prevent “floating” of the instruments in situations such as a leaning position. The tips of the instruments are kept protected as much as possible. Sterility is also safe-guarded. In some embodiments, the open tops of the pockets 120 can have a closure, such as a hook and loop closure, an elastic band, or a purse string.

The pockets 120 can be located at various locations on the gown 100. For example, one or more pockets can be located on the sleeves (above and/or below the elbow), bodice (upper torso area), sides, and/or legs of the gown 100.

The pockets 120 can have various shapes. The gown 100 can have pockets 120 with various shapes. Alternatively, in some embodiments all of the pockets 120 have the same shape on a gown 100. In some embodiments, one or more of the pockets 120 can be specifically shaped to contain a particular type of instrument. In some embodiments, the pocket 120 can comprise a mesh material, transparent material, and/or elastic material.

The pockets 120 and/or loops 110 can be attached directly on the gown 100 when the gown 100 is manufactured. Alternatively, or additionally, in some embodiments a separate patch 130 of fabric that includes one or more loops 110 and/or one or more pockets 120 can be attached to the gown 100 after the manufacturing of the gown 100. Such a separate, attachable patch 130 could be made in various shapes such as, but not limited to, square, rectangular, circular, and elliptical. An attachable patch 130 can be delivered together with the gown 100 as a system, or purchased/available separately from the gown 100.

The base material of the attachable patch 130 can have an adhesive back that could then be attached onto the gown 100 “on demand” in the O.R. as needed. As an alternative to adhesive attachment, in some embodiments the attachable patch 130 can be attached to the gown 100 using hook and loop fastener(s), zipper(s), ties, and the like, and combinations thereof. In some embodiments, the attachable patch 130 can come pre-secured and sealed to the gown 100 during manufacturing for greater weight-bearing security.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying FIGURES do not necessarily require the particular order shown, or sequential order, to achieve desirable results.

What is claimed is:

1. A patch for holding surgical instruments, the patch comprising:
a base material that is attachable to a surgical gown;
one or more pockets attached to the base material and configured to releasably hold surgical instruments; and
one or more rings attached to the base material and configured to releasably hold surgical instruments.

2. The patch of claim 1, wherein the base material includes an adhesive by which the base material is attachable to the surgical gown.

3. A surgical garment system, comprising:
a gown configured to be worn by a human and to be sterile for use in a sterile surgical environment;
one or more pockets attached to the gown and configured to hold surgical instruments;
one or more rings attached to the gown and configured to hold surgical instruments; and
a patch that is attachable to the gown, the patch including one or more additional pockets or additional rings.

4. The surgical gown of claim 3, wherein the patch includes an adhesive by which the patch is attachable to the gown.

5. A surgical garment, comprising:
a gown configured to be worn by a human and to be sterile for use in a sterile surgical environment;
one or more pockets attached to the gown and configured to hold surgical instruments; and
one or more rings attached to the gown and configured to hold surgical instruments,
wherein the one or more pockets includes at least one pocket attached to a sleeve of the gown and at least one pocket attached to a chest area of the gown.

6. The surgical garment of claim 5, wherein the one or more rings includes at least one ring attached to the sleeve of the gown and at least one ring attached to the chest area of the gown.
